(19) United States
(12) Patent Application Publication
(10) Pub. No.: US 2003/0220555 A1
(43) Pub. Date: Nov. 27, 2003

(54) Method and apparatus for image presentation of a medical instrument introduced into an examination region of a patent

(76) Inventors: Benno Heigl, Untersiemau (DE); Joachim Hornegger, Baiersdorf (DE); Reinmar Killmann, Forchheim (DE); Norbert Rahn, Forchheim (DE); Johann Seissl, Erlangen (DE); Siegfried Wach, Hoechstadt (DE)

Correspondence Address: SCHIFF HARDIN & WAITE, 6600 SEARS TOWER, 233 S WACKER DR, CHICAGO, IL, 60606-6473, US

(21) Appl. No.: 10/385,585

(22) Filed: Mar. 11, 2003

(30) Foreign Application Priority Data
Mar. 11, 2002 (DE) 10210647.9

(51) Int. Cl. A61B 5/05

(52) U.S. Cl. 600/407

(57) ABSTRACT

In a method and apparatus for image presentation of a medical instrument introduced into an examination region of a patient, particularly a catheter in the framework of a cardiological examination or treatment, a 3D image dataset of the examination region is employed to generate a 3D reconstruction image of the examination region, at least two 2D fluoroscopic images of the examination region are acquired that reside at an angle relative to one another and wherein the instrument is shown, the 3D reconstruction image is registered relative to the 2D fluoroscopic images, the spatial position of the catheter tip and the spatial orientation of a section of the catheter tip are determined on the basis of the 2D fluoroscopic images; and the 3D reconstruction image is presented at a monitor, this presentation containing a positionally exact presentation of the tip and of the section of the catheter tip of the catheter in the 3D reconstruction image.

### BACKGROUND OF THE INVENTION

1. Field of the Invention

The present invention is directed to a method for image presentation of a medical instrument introduced into an examination region of a patient, particularly a catheter in the framework of a cardiological examination or treatment.

2. Description of the Prior Art

Examinations or treatments of patients are ensuing in minimally invasive fashion to an increasing degree, i.e. with the lowest possible operative outlay. Examples are treatments with endoscopes, laparoscopes or catheters that are each introduced into the examination region of the patient via a small body opening. Catheters are frequently utilized in the framework of cardiological examinations, for example in the case of arrhythmias of the heart that are currently treated by ablation procedures.

Under X-ray supervision, i.e. with the acquisition of fluoroscopic images, a catheter is guided into a heart chamber via veins or arteries. In the heart chamber, the tissue causing the arrhythmia is ablated by applying a high-frequency current, as a result of which the previously arrhythmogenic substrate is left behind as necrotic tissue. The healing nature of this method exhibits significant advantages compared to lifelong medication; moreover, this method is economical in the long view.

A problem from a medical/technical point of view is that although the catheter can be visualized very exactly and highly resolved during the X-ray supervision in one or more fluoroscopic images—also called fluoro images—during the intervention, the anatomy of the patient can be only very inadequately imaged in the fluoroscopic images during the intervention. For tracking the catheter, two 2D fluoroscopic exposures conventionally have been produced from two different projection directions that mainly reside orthogonally relative to one another. On the basis of the information contained in these two exposures, the physician must determine the position of the catheter from the physician's own visual impression, which is often possible only in a relatively imprecise way.

### SUMMARY OF THE INVENTION

An object of the present invention is to provide a presentation that allows the attending physician to make a simple recognition of the exact position of the instrument in the examination region, for example, of a catheter in the heart.

This object is achieved in a method of the type initially described wherein a 3D image dataset of the examination region is employed to generate a 3D reconstruction image of the examination region, at least two 2D fluoroscopic images of the examination region are acquired that reside at a non-zero angle relative to one another and wherein the instrument is shown, the 3D reconstruction image brought into registration relative to the 2D fluoroscopic images, the spatial position of the instrument tip and the spatial orientation of a section of the instrument tip are determined on the basis of the 2D fluoroscopic images, and the 3D reconstruction image is presented at a monitor with a positionally exact presentation of the instrument tip and the section of the instrument tip in the 3D reconstruction image.

The inventive method and apparatus make it possible to display the instrument, i.e. the catheter (only a catheter shall be referred to below) in e three-dimensional presentation of the examination region, for example, of the heart or of a central cardial vessel tree. The presentation occurs quasi in real time during the examination and is exact both as to spatial position as well as spatial orientation. This is possible because a three-dimensional reconstruction presentation of the examination region is generated using a 3D image dataset. Inventively, further, the spatial position of the catheter tip as well as the spatial orientation of a section of the catheter tip, i.e. a section of a specific length of the catheter, is determined starting at the catheter tip. When these coordinates have been acquired, then the this length of the section of the catheter tip is mixed into the 3D reconstruction image with correct position and correct spatial orientation, this being possible since the 3D reconstruction image as well as the two 2D fluoroscopic images are registered relative to one another, i.e. their coordinate systems are correlated with one another via a transformation matrix. The physician is thus shown very exact, spatial orientation information with respect to the catheter, which is shown in its actual position in the examination region. This enables the navigation of the catheter in a simple way since the physician—on the basis of the inventively presented spatial position—can decide in a target-oriented way as to how the instrument must be subsequently moved in order to reach a desired target.

For determining the spatial position of the catheter tip, the tip can be identified in the at least two 2D fluoroscopic images and a back-projection line is subsequently calculated on the basis of the projection matrix of the respective 2D fluoroscopic image, the spatial position being identified on the basis of the back-projection lines. Ideally, the spatial position lies in the intersection of the two projection lines. Due to the structural pre-conditions, which insure the radiation source and the radiation detector do not assume exactly the same position relative to one another in the respective positions at which the fluoroscopic images were acquired, it often occurs that the calculated back-projection lines do not intersect. In such a case, a computational position determination of such a nature ensues to calculate, on the basis of the non-intersecting back-projection lines, a position that comes close to the positions of the tip identified in the 2D fluoroscopic images. For example, an arbitrary point in the given volume can be employed for this purpose, this being changed in position in the course of an optimization process until it comes closest to the identified position of the tip in the 2D fluoroscopic images. As an alternative, it is also possible to determine the middle of the imaginary connecting line between the two back-projection lines at the location of the minimum spacing as the computational position.

In accordance with the invention, the determination of the spatial orientation of the section of the catheter tip ensues by determining an orientation line of a limited length of the catheter tip section in the 2D fluoroscopic images. This orientation line is back-projected to a defined back-projection plane, and the determination of the spatial orientation ensues on the basis of back-projection planes that are generated by the two orientation lines in the respective fluoroscopic images. The physician thus interactively defines this orientation line on the basis of the catheter shown in a fluoroscopic image. This orientation lines describes a section of limited length at the catheter tip, the orientation line corresponding to the orientation of the catheter section in the fluoroscopic image. The back-projection of such an orientation line onto the X-ray tube focus defines a back-projection plane. Two back-projection planes that proceed at an angle to one another thus are obtained, and the spatial orientation can be determined on the basis of these back-projection planes. However, the determination of the orientation line alternatively can ensue automatically.

When two fluoroscopic images are employed for determining the orientation, then the orientation of the catheter tip section is identified on the basis of the line of intersection of the two back-projection planes. Two planes intersect in a straight line. In the inventive method, this straight intersection line exactly specifies the spatial orientation of the catheter tip section in the volume.

When more than two fluoroscopic images are employed wherein respective orientation lines are determined, then the orientation of the catheter tip section can be determined as the straight line that lies closest to the back-projection planes, even though they might not intersect in a shared intersection line. In this case, thus, the conditions are again not ideal, since all projection planes would ideally have to intersect in a shared line. A computational determination of an ideal intersection line that takes the actual courses of the projection planes into consideration ensues for alleviating this situation.

The 3D image dataset can be a pre-operatively acquired dataset. I.e., the dataset can have been acquired at an arbitrary point in time before the actual intervention. Ant 3D image dataset can be employed that is acquired by any acquisition modality, for example, a CT dataset, a MR dataset or a 3D X-ray angiography dataset. All of these datasets allow an exact reconstruction of the examination region, so that this can be displayed anatomically exact and with high resolution. As an alternative, there is the possibility of also employing an intraoperatively acquired dataset in the form of a 3D X-ray angiography dataset. The term “intraoperatively” means that this dataset is acquired in the immediate temporal context of the actual intervention, i.e. when the patient is already lying on the examination table but the catheter has not yet been placed, although this will ensue shortly after the acquisition of the 3D image dataset.

When the examination region is a rhythmically or arrhythmically moving region, for example the heart, then for an exact presentation, the 3D reconstruction image and the 2D fluoroscopic images that are to be acquired must each show the examination region in the same motion phase, or must have been acquired in the same motion phase. In order to enable this, the motion phase can be acquired for the 2D fluoroscopic images, and only the image date that is acquired in the same motion phase as the 2D fluoroscopic images is employed for the reconstruction of the 3D reconstruction image. The acquisition of the motion phase is required in the acquisition of the 3D image dataset as well as in the 2D fluoroscopic image acquisition in order to be able to produce isophase images or volumes. The reconstruction and the image data employed therefor are based on the phase in which the 2D fluoroscopic images were acquired. An ECG that records the heart movements and is acquired in parallel is an example of an acquisition of the motion phase. The relevant image data can then be selected on the basis of the ECG. A triggering of the acquisition device via the ECG can ensue for the acquisition of the 2D fluoroscopic images, so that successively acquired 2D fluoroscopic images are always acquired in the same motion phase. It is also possible to record the respiration phases of the patient as the motion phase. This, for example, can ensue using a respiration belt that is placed around the chest of the patient and measures the movement of the rib cage. Position sensors at the chest of the patient also can be employed for the recording thereof. If the 3D image dataset was already generated with respect to a specific motion phase, then the triggering of the acquisition of the fluoroscopic images is based on the phase of the 3D image dataset.

It is also expedient when, in addition to the motion phase, the point in time of the acquisition of the 2D fluoroscopic images is acquired, and only image data that are also acquired at the same point in time as the 2D fluoroscopic images are employed for the reconstruction of the 3D reconstruction image. The heart changes in shape within a motion cycle of, for example, one second, only within a relatively narrow time window, when it contracts. The heart retains its shape over the rest of the time. Using time as a further dimension, it is then possible to enable a quasi cinematographic, three-dimensional presentation of the heart, since the 3D reconstruction image can be reconstructed for every point in time and correspondingly isochronically acquired 2D fluoroscopic images are present wherein the orientation of the catheter tip can be determined (a bi-plane C-arm apparatus is preferably employed for this purpose). A quasi-cinematographic presentation of the beating heart overlaid with a cinematographic presentation of the guided catheter is obtained as a result. In other words, a separate phase-related and time-related 3D reconstruction image is generated at various points in time with a motion cycle of the heart and a number of phase-related and time-related fluoroscopic images are obtained, with the identified orientation and position of the catheter mixed into the isophase and isochronic 3D reconstruction image, so that the instrument is displayed in the moving heart as a result of successively ensuing output of the 3D reconstruction images and mixing-in of the catheter.

It is especially advantageous for the physician when the common monitor presentation of the 3D reconstruction image with the mixed-in catheter tip and the catheter tip section can be modified by user inputs, particularly rotated, enlarged or reduced, so that the placement of the catheter tip section in the reconstructed organ, for example the heart, can be recognized even more exactly in this way and, for example, its proximity to a cardiac wall can be determined with utmost precision. The catheter tip and the catheter tip section can be presented colored or flashing in order to improve recognition thereof.

Different alternatives are possible for registering the 2D fluoroscopic images with the 3D reconstruction image or the underlying datasets. There is the possibility of employing anatomical picture elements or a number of markings for the aforementioned registration. The registration thus ensues on the basis of anatomical characteristics such as, for example, the heart surface or specific vascular branching points, etc. Instead of employing these anatomical landmarks, however, it is also possible to employ non-anatomical landmarks, i.e. specific markings or the like located in the image that can be recognized in the fluoroscopic images as well as in the 3D reconstruction image. Those skilled in the art are familiar with various registration possibilities that can be utilized in the present method and apparatus. A more detailed discussion thereof is not required. The same is true with regard to generating the 3D reconstruction image. This can be generated in the form of a perspective maximum-intensity projection (MPI) or in the form of a perspective volume-rendering projection image (VRT). Again, those skilled in the art are familiar with various image generating possibilities that can be utilized as needed in the inventive method. This also need not be described in greater detail since these techniques are known to those skilled in the art.

### DESCRIPTION OF THE DRAWINGS

FIG. 1 is a schematic illustration of an inventive medical examination and/or treatment apparatus operable in accordance with the inventive method.

FIG. 2 is a schematic illustration for explaining the spatial position of the catheter tip and the spatial orientation of the catheter tip section in the inventive method and apparatus.

### DESCRIPTION OF THE PREFERRED EMBODIMENTS

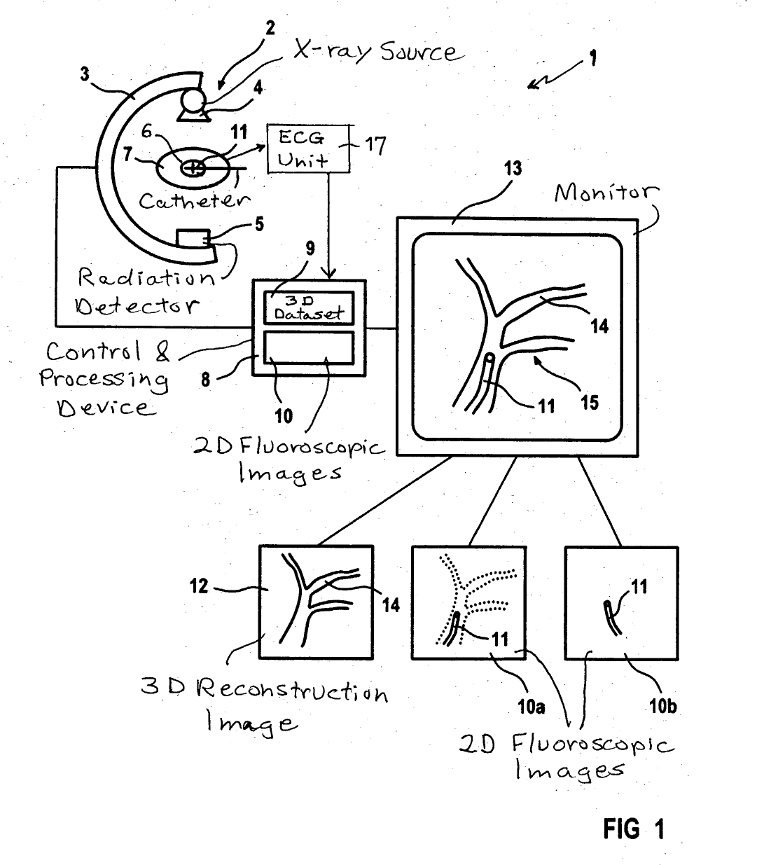

FIG. 1 is a schematic illustration of an inventive examination and/or treatment apparatus 1, with only the basic components being shown. The apparatus has an exposure device 2 for obtaining two-dimensional fluoroscopic images. This is composed of a C-arm 3 at which an X-ray source 4 and a radiation detector 5, for example a solid-state image detector, are arranged. The examination region 6 of a patient is situated essentially in the isocenter of the C-arm, so that the full extent thereof can be seen in the acquired 2D fluoroscopic image.

The operation of the apparatus 1 is controlled by a control and processing device 8 that, among other things, controls the image exposure mode, and which includes an image processor (not shown in detail). A 3D image dataset 9 is present in the device 8, preferably this having been preoperatively acquired. This 3D image dataset can have been acquired with an arbitrary examination modality, for example a computed tomography apparatus or a magnetic resonance apparatus or a 3D angiography apparatus. It can also be acquired as a quasi intraoperative dataset with the installed image exposure device 2, i.e. immediately before the actual catheter intervention, with the image exposure device 2 being for that purpose operated in the 3D angiography mode.

In the illustrated example, a catheter 11 is introduced into the examination region 6, which is the heart in the exemplary embodiment. This catheter can be seen in the 2D fluoroscopic image 10, which is shown enlarged in FIG. 1 in the form of a schematic illustration.

The anatomical environment around the catheter 11, however, cannot be seen in the 2D fluoroscopic image 10. In order to also make this visible, a 3D reconstruction image 12 is generated from the 3D image dataset 9 using known reconstruction methods, this being likewise shown schematically in an enlarged illustration in FIG. 1. This reconstruction image can be generated, for example, as a MIP image or as a VRT image.

The 3D reconstruction image 12 wherein the anatomical environment—a cardial vessel tree 14 in the exemplary embodiment—can be seen is displayed as a three-dimensional image at a monitor 13. As described below, the spatial orientation and position of the catheter tip section are now determined on the basis of two 2D fluoroscopic images residing at an angle relative to one another, preferably residing at a right angle relative to one another. The two fluoroscopic images and the 3D image dataset or and the 3D reconstruction image, are registered (brought into registration) with one another via a transformation matrix. Thus, the catheter 11 is shown in exact position and orientation relative to the vessel tree 14 in the output image 15. On the basis thereof, the physician can recognize exactly where the catheter 11 is located and the physician can determine how the physician must continue to navigate, or how and where the treatment should begin or continue.

The catheter 11 can be shown in an arbitrary emphasized presentation so that it can be recognized clearly and well. For example, it can be boosted in terms of contrast; it can also be displayed in color.

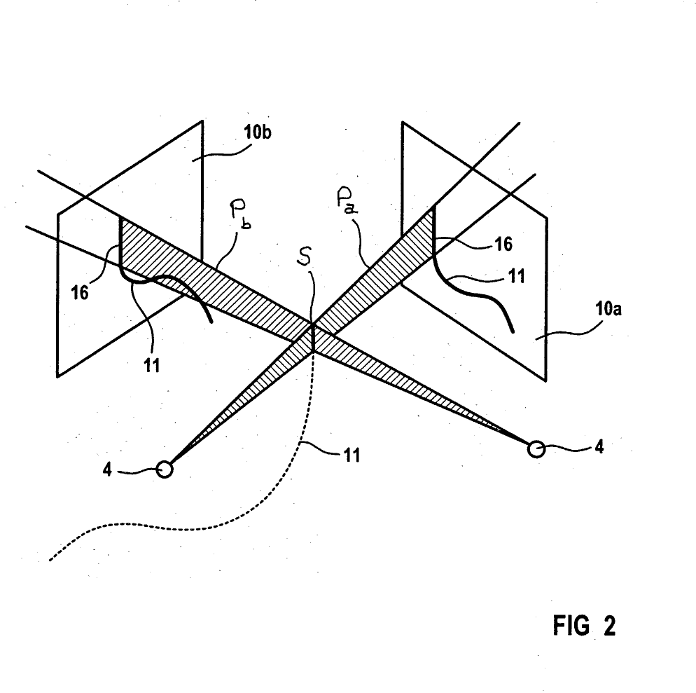

As a schematic diagram, FIG. 2 shows the determination of the spatial orientation of a catheter tip section. Two fluoroscopic images 10a, 10b that were acquired from the examination region residing at a non-zero angle relative to one another, serve this purpose. The image planes preferably reside perpendicularly relative to one another. In the illustrated exemplary embodiment, the catheter 11 is shown in the two 2D fluoroscopic images 10a, 10b. As can be seen, the placement of the catheter 11—which is also shown in FIG. 2 in its spatial position in the examination region (not shown)—differs dependent on the direction from which the examination region and thus the catheter 11 were acquired.

For determining the orientation, an orientation line 16 is defined in each 2D fluoroscopic image 10a, 10b proceeding from the catheter tip, this orientation line 16 indicating the course of the catheter 11 shown in the respective fluoroscopic over a specific section beginning from the catheter tip. The orientation line 16 has a specific length that, for example, can be interactively defined by the physician. Of course, it is also possible that have this orientation line 16 defined automatically by means of a suitable image analysis algorithm.

The orientation line 16 is now projected back over its entire length onto the focus or, respectively, the projection origin of the radiation source 4. Projection planes Pₐ and P_{b} are thereby obtained for the back-projection of an orientation line 16 shown in a fluoroscopic image 10a, 10b. The orientation planes Pₐ, P_{b} intersect along a straight line. This straight line (intersection line S) exactly indicates the spatial orientation of the catheter tip section—which was defined via the orientation line 16—in the examination volume.

The coordinates of the intersection line S, expediently the coordinates of the starting and ending point thereof, are now determined. Due to the registration of the two 2D fluoroscopic images 10a, 10b with the 3D image dataset 9 (and thus with the 3D reconstruction image 12), the intersection lines and consequently the catheter tip section that the intersection line S marks can now be mixed into the three-dimensionally presented examination volume or into the examination region in the 3D reconstruction image 12 with exact position and correct orientation.

The determination of the catheter tip position also can be derived in a simple way from FIG. 2. To this end, the position of the catheter tip is merely identified in the two fluoroscopic images 10a, 10b. The respective positions are then projected back onto the projection origin in the form of a projection line. Two back-projection lines are thus obtained, compared to the two projection planes as employed for the identification of the orientation. In the ideal case, the position of the catheter tip in the three-dimensional examination volume is derived as the intersection of the two projection lines. If these lie somewhat apart, which may be the case due to structural constraints, then the position is computationally determined.

In addition to the possibility of determining the orientation by means of two 2D fluoroscopic images as shown in FIG. 2, it is also possible to employ more than two fluoroscopic images for this purpose. In the ideal case, the number of projection planes that then arise intersect in a common intersection line. If they do not intersect in a common intersection line, then this is likewise identified computationally by means of a suitable approximation to the projection planes.

As described above, if the examination region exhibits a motion phase, the locations in the motion phase, as well as the times, at which the 2D fluoroscopic images were acquired can be identified and only image data in the 3D image dataset 9 are used to generate the 3D reconstruction image 12 that were acquired at the same motion phase locations (and at the same times, if time is also used) as the 2D fluoroscopic images. In the embodiment of FIG. 1 such locations and times are identified from an ECG obtained with an ECG unit 17. The ECG unit 17 is connected to the control and processing device 8 for use thereby in triggering the acquisition of the 2D fluoroscopic images and correlating the image data in the 3D image dataset 9.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for presenting an image of a medical instrument introduced into an examination region of a patient, comprising the steps of:
from a 3D image dataset of an examination region of a patient, generating a 3D reconstruction image of said examination region;
acquiring at least two 2D fluoroscopic images of said examination region, after introducing a medical instrument therein, that reside at a non-zero angle relative to each other and wherein said medical instrument is shown;
bringing said 3D reconstruction image into registration relative to said 2D fluoroscopic images;
determining a spatial position of a tip of said medical instrument and a spatial orientation of a section of said tip from said 2D fluoroscopic images; and
dependent on said determination of said spatial position of said tip and said spatial orientation of said section of said tip, presenting said 3D reconstruction image with a positionally exact presentation of said tip and of said section of said tip in said 3D reconstruction image at a monitor.

2. A method as claimed in claim 1 comprising defining an orientation line having a limited length of said instrument tip for determining the spatial orientation of the section of the tip in the 2D fluoroscopic images by, for each of said 2D fluoroscopic images, back-projecting an image of said section of said tip therein in a back-projection plane, and determining said spatial orientation dependent on the respective back-projection planes.

3. A method as claimed in claim 2 comprising employing two 2D fluoroscopic images and thereby obtaining two back-projection planes, and determining the spatial orientation of said section of said tip by an intersection line of said two back-projection planes.

4. A method as claimed in claim 2 comprising employing more than two 2D fluoroscopic images, and thereby obtaining more than two back-projection planes, and determining the spatial orientation of said section of said tip by defining a straight line lying closest to an intersection of said more than two back-projection planes.

5. A method as claimed in claim 1 comprising determining the spatial position of said tip by, for each of said 2D fluoroscopic images, determining a spatial position of said tip therein and calculating a back-projection line therefrom using a projection matrix for that 2D fluoroscopic image, thereby obtaining at least two back-projection lines, and determining said spatial position from said at least two back-projection lines.

6. A method as claimed in claim 5 wherein said at least two back-projection lines intersect at a point, and defining said spatial position of said tip as said point.

7. A method as claimed in claim 5 wherein said at least two back-projection lines do not intersect, and defining said spatial position of said tip with a computational determination dependent on the respective positions of the tip in said at least two 2D fluoroscopic images.

8. A method as claimed in claim 7 wherein said computational determination comprises selecting an arbitrary point in a volume defined by said nonintersecting back-projection lines, and varying a position of said point in said volume in an optimization process until said point comes closest to correspondence with the respective positions of said tip in said at least two 2D fluoroscopic images.

9. A method as claimed in claim 7 comprising employing two 2D fluoroscopic images and thereby obtaining two non-intersecting back-projection lines, and wherein said computational determination comprises identifying a location of minimum spacing between said two back-projection lines and defining said position of said tip as a mid-point of an imaginary line connecting said two back-projection lines at said location of minimum spacing.

10. A method as claimed in claim 1 comprising acquiring said 3D image dataset of said examination region of said patient before introduction of said medical instrument therein.

11. A method as claimed in claim 1 comprising acquiring said 3D image dataset of said examination region of said patient during introduction of said medical instrument therein.

12. A method as claimed in claim 1 wherein said examination region exhibits movement having a motion phase, and comprising the additional steps of:
acquiring said motion phase;
identifying respective locations in said motion phase at which said at least two 2D fluoroscopic images are acquired; and
employing only image data from said 3D image dataset for reconstructing said 3D reconstruction image acquired at the same respective locations in said motion phase at which said at least 2D fluoroscopic images are acquired.

13. A method as claimed in claim 12 wherein said examination region is a heart and wherein the step of acquiring said motion phase comprises obtaining an ECG of said heart, and identifying the respective same locations in said motion phase, at which said at least two 2D fluoroscopic images and said image data employed for reconstructing said 3D reconstruction image are acquired, from said ECG.

14. A method as claimed in claim 12 comprising the additional steps of:
identifying respective points in time at which said at least two 2D fluoroscopic images are acquired, in addition to said respective locations in said motion phase; and
employing only image data in said 3D image dataset for reconstructing said 3D reconstruction image acquired at the same respective points in time as said at least two 2D fluoroscopic images.

15. A method as claimed in claim 12 wherein said examination region is a heart and wherein the step of acquiring said motion phase comprises obtaining an ECG of said heart, and identifying the respective same times, at which said at least two 2D fluoroscopic images and said image data employed for reconstructing said 3D reconstruction image are acquired, from said ECG.

16. A method as claimed in claim 1 comprising allowing user-entered modifications of said presentation of said 3D reconstruction image with said tip and said section of said tip therein at said monitor.

17. A method as claimed in claim 1 comprising presenting said tip and said section of said tip in said presentation at said monitor using a distinctive presentation characteristic selected from the group consisting of coloring and flashing.

18. An apparatus for presenting an image of a medical instrument introduced into an examination region of a patient:
an image computer for, from a 3D image dataset of an examination region of a patient, generating a 3D reconstruction image of said examination region;
an image acquisition system for acquiring at least two 2D fluoroscopic images of said examination region, after a medical instrument has been introduced therein, that reside at a non-zero angle relative to each other and wherein said medical instrument is shown;
a monitor connected to said image computers; and
said computer bringing said 3D reconstruction image into registration relative to said 2D fluoroscopic images and determining a spatial position of a tip of said medical instrument and a spatial orientation of a section of said tip from said 2D fluoroscopic images, and dependent on said determination of said spatial position of said tip and said spatial orientation of said section of said tip, presenting said 3D reconstruction image with a positionally exact presentation of said tip and of said section of said tip in said 3D reconstruction image at said monitor.

19. An apparatus as claimed in claim 18 wherein said image computer defines an orientation line having a limited length of said tip and determines the spatial orientation of the section of the instrument tip in the 2d fluoroscopic images by, for each of said 2D fluoroscopic images, back-projecting an image of said tip section therein in a back-projection plane, and determining said spatial orientation dependent on the respective back-projection planes.

20. An apparatus as claimed in claim 19 wherein said image acquisition system acquires two 2D fluoroscopic images and said image computer obtains two back-projection planes, and determines the spatial orientation of said section of said tip by an intersection line of said two back-projection planes.

21. An apparatus as claimed in claim 19 wherein said image acquisition system acquires more than two 2D fluoroscopic images, and said image computer obtains more than two back-projection planes, and determining the spatial orientation of said section of said tip by defining a straight line lying closest to an intersection of said more than two back-projection planes.

22. An apparatus as claimed in claim 18 wherein said image computer determines the spatial position of said tip by, for each of said 2D fluoroscopic images, determining a spatial position of said tip therein and calculating a back-projection line therefrom using a projection matrix for that 2D fluoroscopic image, thereby obtaining at least two back-projection lines, and determining said spatial position from said at least two back-projection lines.

23. An apparatus as claimed in claim 22 wherein said at least two back-projection lines intersect at a point, and wherein said image computer defines said spatial position of said instrument tip as said point.

24. An apparatus as claimed in claim 22 wherein said at least two back-projection lines do not intersect, and wherein said image computer defines said spatial position of said instrument tip with a computational determination dependent on the respective positions of the tip in said at least two 2D fluoroscopic images.

25. An apparatus as claimed in claim 24 wherein said image computer in said computational determination selects an arbitrary point in a volume defined by said non-intersecting back-projection lines, and varies a position of said point in said volume in an optimization process until said point comes closest to correspondence with the respective positions of said tip in said at least two 2D fluoroscopic images.

26. An apparatus as claimed in claim 24 wherein said image acquisition system acquires two 2D fluoroscopic images and said image computer obtains two non-intersecting back-projection lines, and wherein said image computer in said computational determination identifies a location of minimum spacing between said two back-projection lines and defines said position of said tip as a mid-point of an imaginary line connecting said two back-projection lines at said location of minimum spacing.

27. An apparatus as claimed in claim 18 wherein said 3D image dataset is a 3D dataset of said examination region of said patient acquired before introduction of said medical instrument therein.

28. An apparatus as claimed in claim 18 wherein said 3D image dataset is a 3D dataset of said examination region of said patient acquired during introduction of said medical instrument therein.

29. An apparatus as claimed in claim 18 wherein said examination region exhibits movement having a motion phase, and comprising:
a unit for acquiring said motion phase; and
wherein said computer identifies respective locations in said motion phase at which said at least two 2D fluoroscopic images are acquired, and employs only image data from said 3D image dataset for reconstructing said 3D reconstruction image acquired at the same respective locations in said motion phase at which said at least 2D fluoroscopic images are acquired.

30. An apparatus as claimed in claim 29 wherein said examination region is a heart and wherein said unit for acquiring said motion phase is an ECG unit which obtains an ECG of the heart, and wherein said image computer identifies the respective same locations in said motion phase, at which said at least two 2D fluoroscopic images and said image data employed for reconstructing said 3D reconstruction image are acquired, from said ECG.

31. An apparatus as claimed in claim 29 comprising:
said image computer identifies respective points in time at which said at least two 2D fluoroscopic images are acquired, in addition to said respective locations in said motion phase, and employs only image data in said 3D image dataset for reconstructing said 3D reconstruction image that are acquired at the same respective points in time as said at least two 2D fluoroscopic images.

32. An apparatus as claimed in claim 29 wherein said examination region is a heart and wherein said unit for acquiring said motion phase is an ECG unit for obtaining an ECG of the heart, and wherein said image computer identifies the respective same times, at which said at least two 2D fluoroscopic images and said image data employed for reconstructing said 3D reconstruction image are acquired, from said ECG.

33. An apparatus as claimed in claim 18 comprising an input unit allowing user-entered modifications of said presentation of said 3D reconstruction image with said tip and said section of said tip therein at said monitor.

34. An apparatus as claimed in claim 18 wherein said image computer presents said tip and said section of said tip in said presentation at said monitor using a distinctive presentation characteristic selected from the group consisting of coloring and flashing.
